(19) United States
(12) Patent Application Publication
(10) Pub. No.: US 2025/0144418 A1
(43) Pub. Date: May 8, 2025

(54) NEUROSTIMULATION CIRCUIT FOR IMPLEMENTATION IN A NEUROSTIMULATION SYSTEM, APPARATUS, AND METHOD

(71) Applicant: AVATION MEDICAL, INC., Columbus, OH (US)

(72) Inventors: Manish Vaishya, Valencia, CA (US); Alexandru Campean, Strongsville, OH (US); Jacob Ryan Sickafoose, Valencia, CA (US); Donald William Rosselot, Cincinnati, OH (US)

(21) Appl. No.: 18/934,455

(22) Filed: Nov. 1, 2024

Related U.S. Application Data
(60) Provisional application No. 63/595,821, filed on Nov. 3, 2023.

(51) Int. Cl. A61N 1/36; A61N 1/02; A61N 1/04

(52) U.S. Cl. CPC A61N 1/36034; A61N 1/025; A61N 1/0484

(57) ABSTRACT

A neurostimulator system for supplying constant current electrical stimulation pulses to at least one stimulation electrode to apply electrical stimulation to a subject includes a power supply comprising a voltage rail and a ground. The system also includes a first constant current circuit configured to supply stimulation current from the voltage rail through the at least one stimulation electrode to ground. A controller is configured to control the constant current circuit. The first constant current circuit includes a first mirror circuit, a first current sink, and a first ground circuit. The first mirror circuit is configured to supply the constant current electrical stimulation pulses from the voltage rail to the at least one electrode. The first current sink is configured to control the constant current electrical stimulation pulses supplied to the at least one electrode from the mirror circuit in response to a first control voltage. The first ground circuit is configured to control a path to ground from the at least one stimulation electrode in response to a first ground control signal from the controller. The controller is configured to control the first control voltage to produce the constant current electrical stimulation pulses and to control the first ground control signal to establish the path to ground so that the constant current electrical stimulation pulses flow through the at least one stimulation electrode in a first direction to apply electrical stimulation to the subject.

### RELATED APPLICATIONS

This application claims the benefit of U.S. Provisional Application Ser. No. 63/595,821, filed on Nov. 3, 2023. This application is hereby incorporated by reference in its entirety.

### TECHNICAL FIELD

The invention relates to a wearable electronic medical device for transcutaneous electrical stimulation of one or more nerves, i.e., neurostimulation.

### BACKGROUND

There are many known technologies for delivering electrical stimulation. Implantable neurostimulation technologies require surgical implantation of stimulation leads, with a pulse generator that is either surgically implanted or connected externally to wire leads. Percutaneous neurostimulation technologies are less invasive, but still require the stimulation electrodes to pierce the skin. While these technologies can be effective in treating certain conditions, they are less desirable due to their invasiveness and because they can require the continued or routine attention of specialists, requiring doctor's office visits, phone calls, etc.

### SUMMARY

A neurostimulation system for applying transcutaneous electrical neurostimulation includes an electronic stimulator device or controller that controls the delivery of transcutaneous electrical neurostimulation signal via stimulation electrodes in contact with the subject's skin. The controller employs a novel circuit for modulating the electrical neurostimulation signal.

The neurostimulator system supplies constant current electrical stimulation pulses to at least one stimulation electrode to apply electrical stimulation to a subject. The system includes a power supply comprising a voltage rail and a ground. The system also includes a first constant current circuit configured to supply stimulation current from the voltage rail through the at least one stimulation electrode to ground. A controller is configured to control the constant current circuit. The first constant current circuit includes a first mirror circuit, a first current sink, and a first ground circuit. The first mirror circuit is configured to supply the constant current electrical stimulation pulses from the voltage rail to the at least one electrode. The first current sink is configured to control the constant current electrical stimulation pulses supplied to the at least one electrode from the mirror circuit in response to a first control voltage. The first ground circuit is configured to control a path to ground from the at least one stimulation electrode in response to a first ground control signal from the controller. The controller is configured to control the first control voltage to produce the constant current electrical stimulation pulses and to control the first ground control signal to establish the path to ground so that the constant current electrical stimulation pulses flow through the at least one stimulation electrode in a first direction to apply electrical stimulation to the subject.

According to one aspect, the controller can be configured to control the first control voltage to cause the first current sink so that the first mirror circuit produces the constant current stimulation pulses while isolating the subject from the voltage rail.

According to another aspect, alone or in combination with the preceding aspect, the first current sink and the current mirror can be electrically connected to one side of the at least one electrode, and the first ground circuit can be connected on an opposite side of the at least one electrode.

According to another aspect, alone or in combination with one or more of the preceding aspects, the first ground control signal can include the first control voltage, and the first ground circuit can include a current sink configured to control a path to ground from the at least one stimulation electrode in response to the first control voltage.

According to another aspect, alone or in combination with one or more of the preceding aspects, the first ground control signal can include a digital control signal, and the first ground circuit can include a MOSFET configured to control a path to ground from the at least one stimulation electrode in response to the digital control signal.

According to another aspect, alone or in combination with one or more of the preceding aspects, the system can include a second constant current circuit including a second mirror circuit configured to supply the constant current electrical stimulation pulses from the voltage rail to the at least one electrode. The second constant current circuit can also include a second current sink configured to control the constant current electrical stimulation pulses supplied to the at least one electrode from the mirror circuit in response to a second control voltage. The second constant current circuit can further include a second ground circuit configured to control a path to ground from the at least one stimulation electrode in response to a second ground control signal from the controller. The controller can be configured to control the second control voltage to produce the constant current electrical stimulation pulses and to control the second ground control signal establish the path to ground so that the constant current electrical stimulation pulses flow through the at least one stimulation electrode in a second direction, opposite the first direction, to apply electrical stimulation to the subject.

According to another aspect in combination with the preceding aspect, the first current sink and the first current mirror can be electrically connected to a first side of the at least one electrode, and the first ground circuit can be connected to a second side of the at least one electrode, opposite the first side. The second current sink and the second current mirror can be electrically connected to the second side of the at least one electrode, and the second ground circuit can be connected to the first side of the at least one electrode.

According to another aspect, alone or in combination with one or more of the preceding aspects, the first ground control signal can include the first control voltage, and the first ground circuit can include a current sink configured to control a path to ground from the at least one stimulation electrode in response to the first control voltage. The second ground control signal can include the second control voltage, and the second ground circuit can include a current sink configured to control a path to ground from the at least one stimulation electrode in response to the second control voltage.

According to another aspect, alone or in combination with one or more of the preceding aspects, the first ground control signal can include a first digital control signal, and the first ground circuit can include a MOSFET configured to control a path to ground from the at least one stimulation electrode in response to the first digital control signal. The second ground control signal can include a second digital control signal, and the second ground circuit can include a MOSFET configured to control a path to ground from the at least one stimulation electrode in response to the second digital control signal.

According to another aspect, alone or in combination with one or more of the preceding aspects, the controller can be configured to enforce a control regimen in which the second control voltage is held at zero while the first control voltage is modulated to provide the constant current electrical stimulation pulses. The controller can also be configured to enforce a control regimen in which the first control voltage is held at zero while the second control voltage is modulated to provide the constant current electrical stimulation pulses.

According to another aspect, alone or in combination with one or more of the preceding aspects, the system can include a wearable upon which the controller and the one or more stimulation electrodes are mounted.

### DRAWINGS

FIG. 1 illustrates a system for delivering transcutaneous neurostimulation to a subject.

FIG. 2 is simplified schematic illustration of a H-bridge switching circuit that can be implemented by the system of FIG. 1.

FIG. 3 is schematic illustration of a Howland current pump circuit that can be implemented by the system of FIG. 1.

FIG. 4 is a schematic illustration of an isolate high-voltage power supply circuit that can be implemented by the system of FIG. 1.

FIG. 5 is schematic illustration of a neurostimulation circuit implemented by the system of FIG. 1.

FIG. 6 is schematic illustration identifying portions of the neurostimulation circuit of FIG. 5.

FIG. 7 is schematic illustration of another neurostimulation circuit implemented by the system of FIG. 1.

### DESCRIPTION

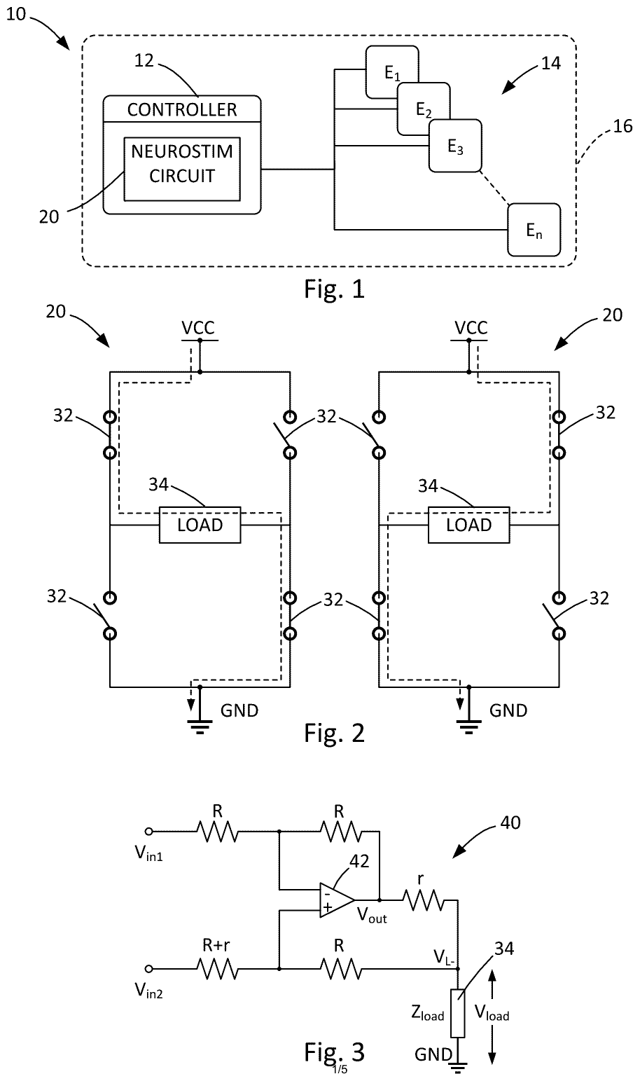

FIG. 1 illustrates by way of example a system 10 for delivering transcutaneous neurostimulation to a subject. The neurostimulation system 10 includes an apparatus in the form of a controller 12 (e.g., a microcontroller) and one or more electrodes 14 (E₁, E₂, E₃, . . . . Eₙ) configured to be positioned on a skin surface. The positioning of the electrodes 14 on the skin surface can be achieved in a variety of manners. For example, the electrodes 14 can be manually positioned on the skin surface, such as by placing stick-on disposable electrodes directly on the skin. In other implementations, the system 10 can include a wearable 16, such as a strap, brace, sock, sleeve, wrap, or garment, upon which the electrodes 14 and/or the controller 12 can be mounted. In this configuration, the electrodes can be positioned in contact with the skin surface when the wearable 16 is placed on the subject.

The system 10 is configured to apply electrical stimulation signals to one or more nerves of the subject through the skin according to a prescribed neurostimulation method. While these methods can vary widely, they all entail varying or modulating the applied electrical neurostimulation signal, a process referred to herein as neuromodulation. To do so, a neurostimulation circuit 20 embedded in the controller 12 includes a constant current source that is controlled to produce the neuromodulation signal. The neurostimulation circuit 20 is capable of high voltage biphasic output applied across a load which, in the neurostimulation setting, includes the stimulated tissue. To accomplish this, three distinct and often contradictory requirements must be managed: 1) minimizing current fluctuation, 2) managing a high rail voltage, and 3) ensuring charge balance in the bi-phasic waveform.

### DC to AC Signal

For neuromodulation, a biphasic signal is preferred so as to avoid charge build up in the subject, thus making the therapy more comfortable and avoiding redux reactions affecting the electrodes 14. It is common for microcontrollers to have built in digital to analog converters (DACs) that allow for an adjustable amplitude voltage output. The problem with this output is that the current flow is limited to one direction. A common solution to adapt this direct current (DC) signal to alternating current (AC) is to utilize solid state switches to alternate the direction of current flow through the output. This is a standard circuit known as an H-bridge, which is shown in FIG. 2.

As shown in FIG. 2, the neurostimulation circuit 20 can include an H-bridge 30, which includes solid state switches 32 that the controller 12 can actuate to control current flow through the load 34, which includes the electrode(s) 14 and the skin/tissue upon which the electrode(s) are applied. In doing so, as shown in FIG. 2, the current flow direction can be changed, so that the neuromodulation can be applied using an alternating current.

### Embedded System Controlled Constant Current Source

Constant current output is important for neuromodulation so that therapy is not affected by potential impedance variations, as are commonly encountered during any real-world subject use. Circuits configured to vary the stimulation voltage in order to maintain a constant stimulation current are sometimes used. A limitation, however, of these circuits is that the current output is limited to one direction of current flow. If this varied voltage constant current neurostimulation signal is applied via an H-bridge (see, FIG. 2) in order to switch the direction of current flow, the H-bridge will disrupt the constant aspect of the current, which implies that the current will vary with the load impedance.

There are standard circuits that can create a biphasic and adjustable amplitude constant current using DC signals. One example is the Howland current pump 40 shown in FIG. 3. Referring to FIG. 3, by outputting voltage to Vᵢₙ₁ and leaving Vᵢₙ₂ as 0V, a constant current flows one way through the load 34 (Z_{load}). Switching Vᵢₙ₁ and Vᵢₙ₂ voltages causes a constant current to flow the opposite way through the load. By using an operational amplifier (op-amp) 42 to supply this current, backflow through Vᵢₙ is prevented which protects the microcontroller.

The problem with this constant current circuit is that neuromodulation requires a high voltage output. The constant current source current pump 40 is directly powered from the op-amp 42, and high voltage capable op-amps are very difficult to design.

### High Voltage Capability

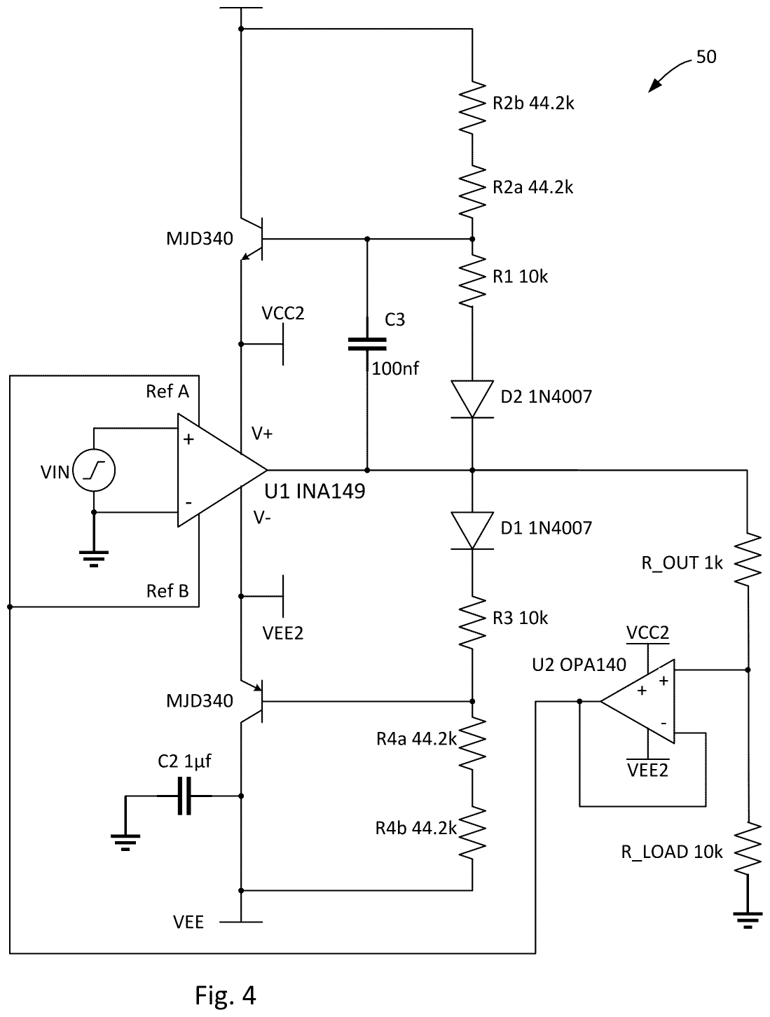

It is possible to isolate the current source from the load and provide the high voltage required to maintain the current separately from the op-amp. An example of such an isolated high-voltage circuit is shown in FIG. 4. As illustrated in FIG. 4, this circuit 50 is increasingly complex and can, for example, require two separate high voltage supplies. As a result, this would, for example, require a voltage differential on the board of 400V to achieve a separate +200V and −200V rail.

### The Neurostimulation Circuit

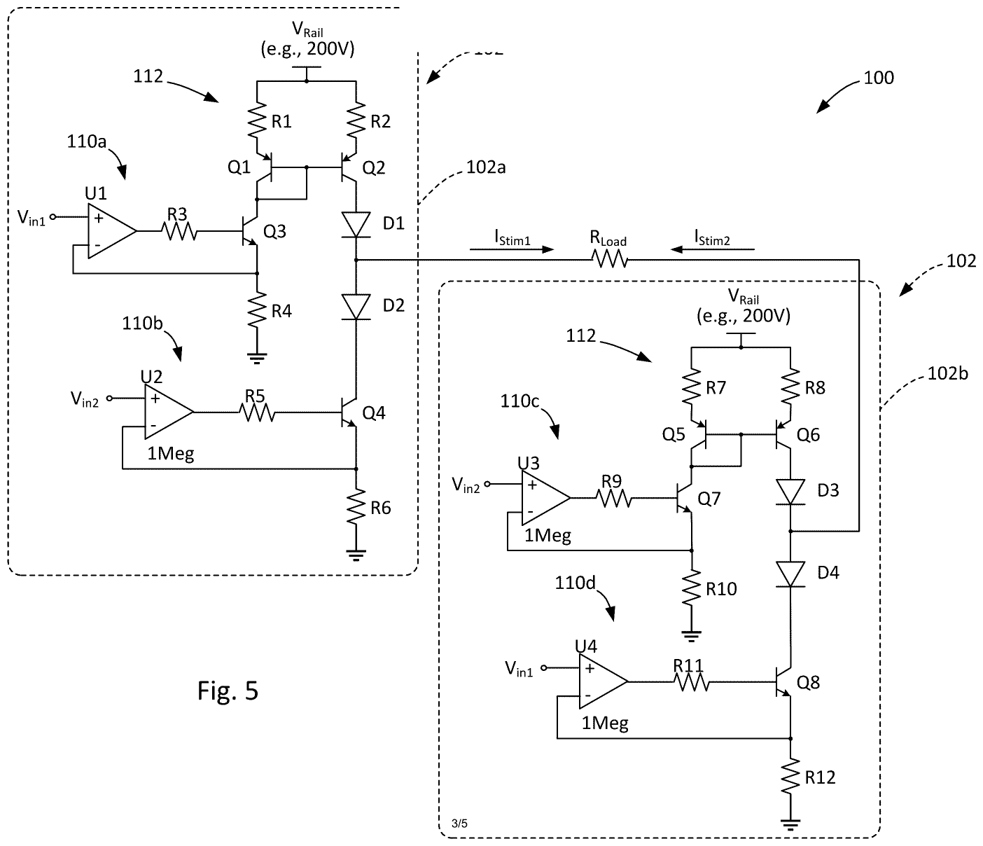

The system 10 implements a neurostimulation circuit that produces a controlled waveform implementing all neurostimulation requirements while utilizing a single high voltage power rail. FIG. 5 shows the neurostimulation circuit 100 for generating the required stimulation waveform with a biphasic constant current signal. The neurostimulation circuit 100 is implemented in the circuitry 20 of the controller 12 of the system 10, and is used to deliver stimulation current to the subject via the electrode(s) 14.

The neurostimulation circuit 100 includes two constant current circuits 102 configured to apply a constant current stimulation signal through the load R_{Load}, i.e., the electrode 14 and the stimulated tissue. A first one of the constant current circuits 102a is configured to apply a constant current stimulation signal I_{Stim1} supplied by the rail voltage V_{Rail} in a first direction through the load R_{Load}, i.e., left-to-right as viewed in FIG. 5. A second one of the constant current circuits 102b is configured to apply a constant current stimulation signal I_{Stim2} supplied by the rail voltage V_{Rail} in a second direction, opposite the first direction, through the load R_{Load}, i.e., right-to-left as viewed in FIG. 5. The controller 12 is configured to control the neurostimulation circuit 100 by modulating or otherwise controlling the control voltages Vᵢₙ₁ and Vᵢₙ₂., which control the operation of the constant current circuits 102.

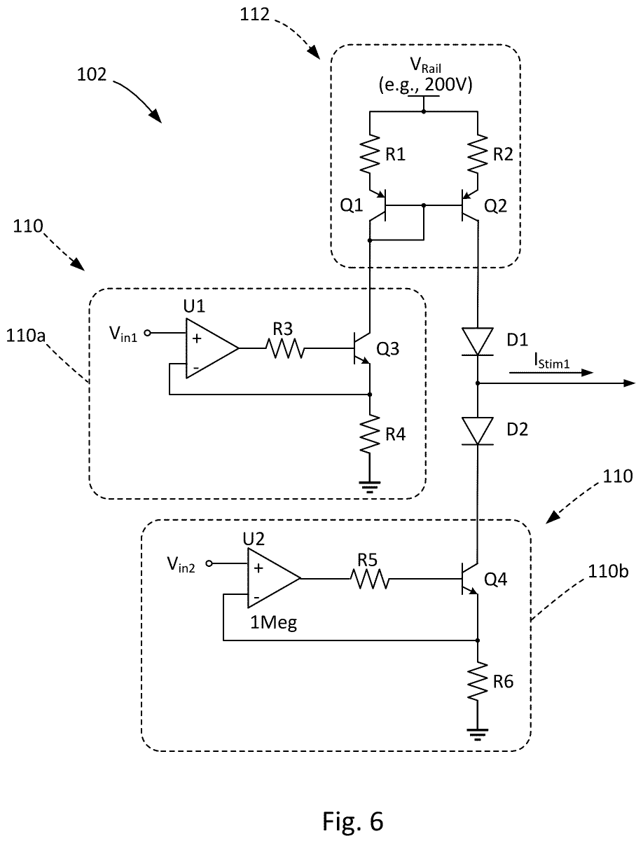

The constant current circuits 102 combine several simple component circuits in a novel way to create a simple and effective solution to the problems outlined above. FIG. 6 illustrates one constant current circuit 102, specifically the first constant current circuit 102a, delineated with dashed-line boxes to identify the component circuits that are combined to produce it. The first of these component circuits that make up the constant current circuits 102 are current sink circuits. As shown in FIG. 6, each constant current circuit 102 includes two current sink circuits 110. Because the current must be supplied from the high voltage power supply rail V_{Rail}, it is necessary to use a current sink circuit, rather than a current source circuit, so that the correct amount of stimulation current I_{Stim1} is drawn from the sourcing voltage rail V_{Rail}. The current sink circuits 110 are grounded, which allows the stimulation current to be controlled at a constant rate despite varying impedances at the load R_{Load}. The current sink circuits 110 are precise, simple, and adjust quickly to impedance variance at the load R_{Load}.

Utilizing the current sink 110 alone would connect the subject directly to a high voltage power supply, i.e. V_{Rail}. Additionally, utilizing the current sink 110 alone would also make it difficult to reverse the current flow through the load R_{Load} due, for example, to the rise and fall times of the transistors Q3 and Q4, which affect the switching speed of the circuits.

To combat this, each constant current circuit 102 also includes an inverse current mirror circuit 112. The mirror circuit 112 isolates the subject from the high voltage rail V_{Rail}. Combining both the current sinks 110 and the mirror circuit 112 provides a novel circuit that enables precise control of current flow with a high maximum voltage, while isolating the subject from the high voltage rail V_{Rail}. The neurostimulation circuit 100 utilizes two of these constant current circuits 102 that can selectively open/close a path to ground in order to generate and maintain the same current flow in both directions through the load R_{Load}. Implementing two constant current circuits 102 as shown in FIG. 6 allows stimulation current to flow both ways through the load R_{Load} while only using one high voltage power supply rail.

Referring to FIG. 6, the two current sink circuits 110 implemented in each of the constant current circuits 102 perform different functions. A first current sink circuit 110a is configured to control stimulation current I_{Stim1} delivered from the rail voltage V_{Rail}. A second current sink circuit 110b is configured for grounding control.

Referring to FIG. 5, to maintain the stimulation current at a constant level, the controller 12 is configured to adjust the control voltages Vᵢₙ₁, Vᵢₙ₂ of the first current sink circuit 110a to cause a corresponding adjustment to the stimulation voltages V_{Stim1}, V_{Stim2} delivered from the rail voltage V_{Rail}. The controller 12 adjusts the control voltages Vᵢₙ₁, Vᵢₙ₂ to produce a corresponding adjustment to the stimulation voltage V_{Stim1}, V_{Stim2} that maintains the constant simulation current I_{Stim1}, I_{Stim2} delivered through the load R_{Load}.

As shown in FIG. 5, the two constant current circuits 102a and 102b implemented in the neurostimulation circuit 100 are identical, except that the control voltages Vᵢₙ₁ and Vᵢₙ₂ are reversed. The first constant current circuit 102a delivers stimulation current through the load R_{Load} in a first direction, as indicated generally by the arrow labeled I_{Stim1} in FIG. 5. The stimulation current I_{Stim1} is controlled by adjusting the control voltage Vᵢₙ₁. The second constant current circuit 102b delivers stimulation current through the load R_{Load} in a second direction, opposite the first direction, as indicated generally by the arrow labeled I_{Stim2} in FIG. 5. The stimulation current I_{Stim2} is controlled by adjusting the control voltage Vᵢₙ₂.

Noting that the neurostimulation circuit 100 implements two constant current circuits 102a, 102b configured to apply stimulation current in opposite directions through the load, it can be seen that when one constant current circuit is supplying stimulation current to the load, the other constant current circuit supplies no current. Thus, the controller 12 is configured so that when the first control voltage Vᵢₙ₁ is non-zero, meaning that the first constant current circuit 102a is supplying the stimulation current I_{Stim1}, the second control voltage Vᵢₙ₂ is zero. Conversely, the controller 12 is configured so that when the second control voltage Vᵢₙ₂ is non-zero, meaning that the first constant current circuit 102b is supplying the stimulation current I_{Stim2}, the first control voltage Vᵢₙ₁ is zero.

Given that the controller 12 is configured so that, during stimulation operation, one control voltage is non-zero and the other is zero, the operation of the constant current circuits 102a, 102b can be seen. When the controller 12 utilizes the first constant current circuit 102a to supply the stimulation current I_{Stim1}, Vᵢₙ₁ is non-zero and Vᵢₙ₂ is zero. Thus, current sink circuit 110a generates a stimulation voltage V_{Stim1}, supplied from the rail voltage V_{Rail}, to produce the desired stimulation current I_{Stim1} through the load R_{Load}, as dictated by the first control voltage Vᵢₙ₁. At the same time, current sink 110d, being controlled by the first control voltage Vᵢₙ₁, provides a path to ground for the stimulation current I_{Stim1}. Also at the same time, while stimulation current is being supplied by the first constant current circuit 102a, because the second control voltage Vᵢₙ₂ is by definition zero, current sink circuit 110b leaves its path to ground open, and current sink circuit 110c produces zero stimulation voltage.

Conversely, when the controller 12 utilizes the second constant current circuit 102b to supply the stimulation current I_{Stim2}, Vᵢₙ₂ is non-zero and Vᵢₙ₁ is zero. Thus, current sink circuit 110c generates a stimulation voltage V_{Stim2}, supplied from the rail voltage V_{Rail}, to produce the desired stimulation current I_{Stim2} through the load R_{Load}, as dictated by the second control voltage Vᵢₙ₂. At the same time, current sink 110b, being controlled by the second control voltage Vᵢₙ₂, provides a path to ground for the stimulation current I_{Stim2}. Also at the same time, while stimulation current is being supplied by the second constant current circuit 102b, because the first control voltage Vᵢₙ is by definition zero, current sink circuit 110d leaves its path to ground open, and current sink circuit 110a produces zero stimulation voltage.

In the neurostimulation circuit 100, the amplitude of voltages Vᵢₙ₁ and Vᵢₙ₂ control the current supply flowing from the rail V_{Rail} through the load R_{Load}. R_{Load} includes the electrode 14 and the tissue of the subject upon which the electrode is positioned. R_{Load} can therefore fluctuate from session to session, and even during the same session due, for example, to the electrode 14 shifting and/or subject sweating. Advantageously, the neurostimulation circuit 100 can maintain a constant current flow through the load R_{Load}, even when the load fluctuates.

### MOSFET Implementation

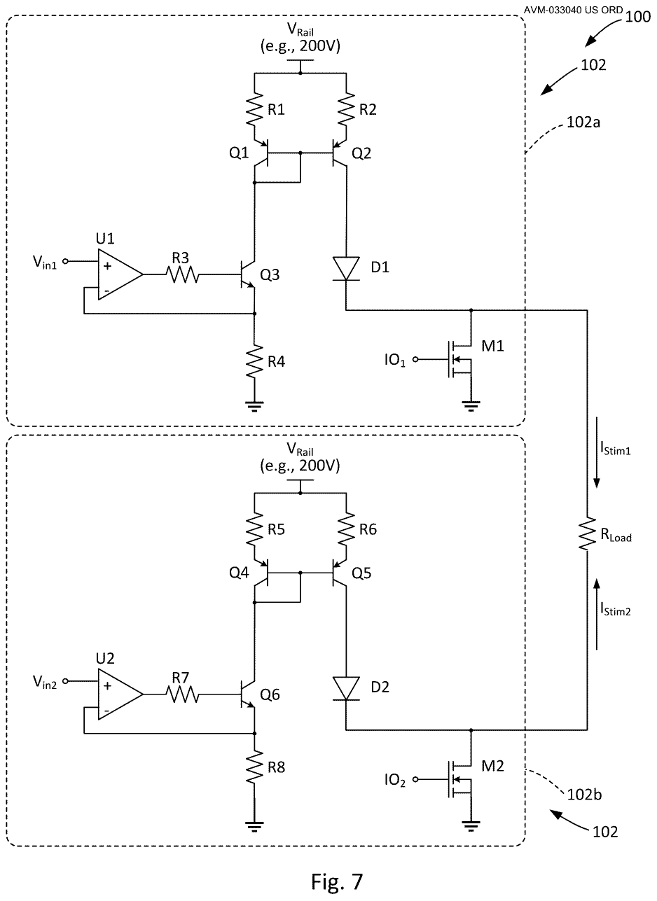

A modification to the neurostimulation circuit 100 is shown in FIG. 7. This neuromodulation circuit 100 utilizes MOSFETs to selectively connect the opposite side of the R_{Load} to ground. This helps simplify the constant current circuits 102, by replacing two of the current sink circuits 110b, 110d with MOSFETs M1, M2, respectively. The neurostimulation circuit 100 maintains the bi-directional current flow capabilities and the de-coupling/isolation of the subject form the rail voltage V_{Rail}. The MOSFET M2 connects the side of the load R_{Load} opposite the constant current circuit 102a to ground. Conversely, the MOSFET M1 connects the side of the load R_{Load} opposite constant current circuit 102b to ground. The control and operation of the MOSFETs M1 and M2 lends a simple and straightforward control for establishing the ground connection necessary for the constant current circuits 102a, 102b to supply current pulses through the load R_{Load}.

Thus, for example, in FIG. 7, to establish a current pulse I_{Stim1} through the load R_{Load}, the controller 12 adjusts the control first voltage Vᵢₙ₁ to a non-zero voltage in order to apply a first stimulation voltage V_{Stim1} from the rail voltage V_{Rail} sufficient to produce the first current pulse I_{Stim1} through the load R_{Load}. At the same time, the controller 12 commands IO₂ to digital 1, closing the path to ground through MOSFET M2. Additionally, the controller 12 controls the second control voltage Vᵢₙ₂ to zero and commands IO₁ to digital 0, opening the path to ground through MOSFET M1.

Conversely, to establish a current pulse I_{Stim2} through the load R_{Load}, the controller 12 adjusts the control second voltage Vᵢₙ₂ to a non-zero voltage in order to apply a second stimulation voltage V_{Stim2} from the rail voltage V_{Rail} sufficient to produce the second current pulse I_{Stim2} through the load R_{Load}. At the same time, the controller 12 commands IO₁ to digital 1, closing the path to ground through MOSFET M1. Additionally, the controller 12 controls the first control voltage Vᵢₙ₁ to zero and commands IO₂ to digital 0, opening the path to ground through MOSFET M2.

From the above, it will be appreciated that the neurostimulation circuits 100 of FIGS. 5-7 combine current sink and current mirror circuits to implement a high voltage capability and voltage controlled constant current source. Utilizing two separate constant current sources on either side of a load, the neurostimulation circuits 100 implement bidirectional current flow with a single high voltage rail. The software implemented in the controller 12 produces a bi-directional constant current source that is capable of outputting high voltages by utilizing two current sink and current mirror systems.

The neurostimulation circuit 100 can be implemented in the neurostimulation system 10 to apply electrical stimulation via the stimulation electrodes 14, which apply the electrical stimulation transcutaneously, according to a proscribed treatment regimen. The neurostimulation circuit can be used to energize the stimulation electrodes in any system, apparatus, or method that implements a neurostimulation treatment or therapy. Implementation of the neurostimulation circuit 100 is not limited to any particular neurostimulation system, apparatus, or method. The neurostimulation circuit 100 can be applied to any system, apparatus, or method for applying electrical neurostimulation. Moreover, implementation of the neurostimulation circuit 100 is not limited to transcutaneous electrical neurostimulation systems, apparatuses, and methods. The neurostimulation circuit can, for example, be implemented in percutaneous electrical neurostimulation systems, apparatuses, and methods.

In one example implementation, the neurostimulation circuit 100 can be implemented in an electronic medical device, a system including the medical device, and a method for using the medical device, configured to apply transcutaneous electrical stimulation to peripheral nerves. According to one specific example, the neurostimulation circuit 100 can be implemented in a system, apparatus, or method for stimulating the tibial nerve (transcutaneous tibial nerve stimulation “TTNS”) to treat medical conditions associated with pelvic floor dysfunction, e.g., over-active bladder (OAB). In a TTNS implementation, a system including an electronic medical device can implement the neurostimulation circuit 100 to apply electrical stimulation near the medial malleolus, which activates both sensory and motor fibers in the nerve. The activation of the sensory fibers of the tibial nerve helps to treat the urge-related symptoms of OAB. The activation of the motor fibers can, however, cause unwanted side effects, such as toe twitch or spasm.

As another example, the system can be used to apply electrical stimulation to the tibial nerve to treat sexual dysfunction. In this manner, it is believed that tibial nerve stimulation could be used to treat genital arousal aspects of female sexual interest/arousal disorder by improving pelvic blood flow.

As another example, the system can be applied to the wrist area to provide stimulation to the ulnar nerve and/or median nerve for pain management in carpal tunnel syndrome, hypertension management, and nerve conduction study/nerve injury diagnosis for median/ulnar nerve neuropathy, etc.

The system and/or the device employed by the system can have a variety of implementations. According to one implementation, the electronic medical device (i.e., the electrodes, control unit, wiring, etc.) can be fixed to a garment that is worn by the subject. The garment can be tight or snug-fitting so as to maintain sufficient contact between the subject's skin and can be configured to position the electrodes at locations specific to the peripheral nerves being stimulated. For example, to stimulate peripheral nerves in the area of the foot or ankle, such as the tibial nerve near the medial malleolus as described above, the garment can be in the form of a sock, ankle brace, strap, sleeve, or other like structure. For stimulating peripheral nerves on the leg, the garment can be a brace, strap, or sleeve sized appropriately for lower leg, knee, or upper leg positioning. For knee or ankle positioning, the garment can be configured, e.g., with openings, slots, or interconnected sections, to allow for bending with the joint while maintaining electrode positioning and contact.

Similarly, for stimulating peripheral nerves on the hand, the garment can be in the form of a glove, mitten, hand brace, or sleeve. For stimulating peripheral nerves on the arm, the garment can be a tight/snug fitting brace, strap, or sleeve (e.g., neoprene) that is sized appropriately for lower arm (forearm/wrist), elbow, or upper arm positioning. For wrist and/or elbow positioning, the sleeve can be configured, e.g., via openings, slots, or interconnected sections, to allow for bending with the joint while maintaining electrode positioning and contact.

In keeping with the above, it will be appreciated that the manner in which the electronic medical device can be secured or supported on the subject can vary. It will also be appreciated that the manner in which the electronic medical device is supported is not critical, as long as contact between the electrodes and the subject's skin is maintained, the positions of the electrode on the subject are maintained, and that the aforementioned are achieved in a manner that is comfortable to the subject.

Various examples of the disclosure have been described. These and other examples are within the scope of the following claims.

We claim:

1. A neurostimulator system for supplying constant current electrical stimulation pulses to at least one stimulation electrode to apply electrical stimulation to a subject, the neurostimulator system comprising:
a power supply comprising a voltage rail and a ground;
a first constant current circuit configured to supply stimulation current from the voltage rail through the at least one stimulation electrode to ground; and
a controller configured to control the constant current circuit;
wherein the first constant current circuit comprises:
a first mirror circuit configured to supply the constant current electrical stimulation pulses from the voltage rail to the at least one electrode;
a first current sink configured to control the constant current electrical stimulation pulses supplied to the at least one electrode from the mirror circuit in response to a first control voltage; and
a first ground circuit configured to control a path to ground from the at least one stimulation electrode in response to a first ground control signal from the controller;
wherein the controller is configured to control the first control voltage to produce the constant current electrical stimulation pulses and to control the first ground control signal to establish the path to ground so that the constant current electrical stimulation pulses flow through the at least one stimulation electrode in a first direction to apply electrical stimulation to the subject.

2. The neurostimulator system recited in claim 1, wherein the controller is configured to control the first control voltage to cause the first current sink so that the first mirror circuit produces the constant current stimulation pulses while isolating the subject from the voltage rail.

3. The neurostimulator system recited in claim 1, wherein the first current sink and the current mirror are electrically connected to one side of the at least one electrode, and the first ground circuit is connected on an opposite side of the at least one electrode.

4. The neurostimulator system recited in claim 1, wherein the first ground control signal comprises the first control voltage, and the first ground circuit comprises a current sink configured to control a path to ground from the at least one stimulation electrode in response to the first control voltage.

5. The neurostimulator system recited in claim 1, wherein the first ground control signal comprises a digital control signal, and the first ground circuit comprises a MOSFET configured to control a path to ground from the at least one stimulation electrode in response to the digital control signal.

6. The neurostimulator system recited in claim 1, further comprising a second constant current circuit comprising:
a second mirror circuit configured to supply the constant current electrical stimulation pulses from the voltage rail to the at least one electrode;
a second current sink configured to control the constant current electrical stimulation pulses supplied to the at least one electrode from the mirror circuit in response to a second control voltage; and
a second ground circuit configured to control a path to ground from the at least one stimulation electrode in response to a second ground control signal from the controller;
wherein the controller is configured to control the second control voltage to produce the constant current electrical stimulation pulses and to control the second ground control signal establish the path to ground so that the constant current electrical stimulation pulses flow through the at least one stimulation electrode in a second direction, opposite the first direction, to apply electrical stimulation to the subject.

7. The neurostimulator system recited in claim 6, wherein:
the first current sink and the first current mirror are electrically connected to a first side of the at least one electrode, and the first ground circuit is connected to a second side of the at least one electrode, opposite the first side; and
the second current sink and the second current mirror are electrically connected to the second side of the at least one electrode, and the second ground circuit is connected to the first side of the at least one electrode.

8. The neurostimulator system recited in claim 5, wherein:
the first ground control signal comprises the first control voltage, and the first ground circuit comprises a current sink configured to control a path to ground from the at least one stimulation electrode in response to the first control voltage; and
the second ground control signal comprises the second control voltage, and the second ground circuit comprises a current sink configured to control a path to ground from the at least one stimulation electrode in response to the second control voltage.

9. The neurostimulator system recited in claim 5, wherein:
the first ground control signal comprises a first digital control signal, and the first ground circuit comprises a MOSFET configured to control a path to ground from the at least one stimulation electrode in response to the first digital control signal; and
the second ground control signal comprises a second digital control signal, and the second ground circuit comprises a MOSFET configured to control a path to ground from the at least one stimulation electrode in response to the second digital control signal.

10. The neurostimulator system recited in claim 6, wherein:
the controller is configured to enforce a control regimen in which the second control voltage is held at zero while the first control voltage is modulated to provide the constant current electrical stimulation pulses; and
the controller is configured to enforce a control regimen in which the first control voltage is held at zero while the second control voltage is modulated to provide the constant current electrical stimulation pulses.

11. The neurostimulator system recited in claim 1, further comprising a wearable upon which the controller and the one or more stimulation electrodes are mounted.
